(19) United States
(12) Patent Application Publication
(10) Pub. No.: US 2016/0100822 A1
(43) Pub. Date: Apr. 14, 2016

(54) BEAMFORMING APPARATUS AND ULTRASOUND DIAGNOSTIC APPARATUS HAVING THE SAME

(71) Applicant: SAMSUNG ELECTRONICS CO., LTD., Suwon-si (KR)

(72) Inventors: Baehyung KIM, Yongin-si (KR); Kyuhong KIM, Seoul (KR); Suhyun PARK, Hwaseong-si (KR); Seungheun LEE, Seongnam-si (KR); Hotaik LEE, Yongin-si (KR); Kyungil CHO, Seoul (KR)

(73) Assignee: SAMSUNG ELECTRONICS CO., LTD., Suwon-si (KR)

(21) Appl. No.: 14/877,376

(22) Filed: Oct. 7, 2015

(30) Foreign Application Priority Data
Oct. 8, 2014 (KR) 10-2014-0135620

(51) Int. Cl. A61B 8/00; A61B 8/14

(52) U.S. Cl. CPC A61B 8/4483; A61B 8/14; A61B 8/54

(57) ABSTRACT

A beamforming apparatus configured to beamform ultrasound waves transmitted through an ultrasound transducer having a two-dimensional transducer array includes a transmitter configured to output transmission pulses configured to drive elements constituting the transducer array, and a transmission switch configured to select at least two elements among the elements to form an aperture such that the transmission pulses drive the elements forming the aperture.

### CROSS-REFERENCE TO RELATED APPLICATION

This application claims the benefit of Korean Patent Application No. 10-2014-135620, filed on Oct. 8, 2014 in the Korean Intellectual Property Office, the disclosure of which is incorporated herein by reference in its entirety.

### BACKGROUND

1. Field

Apparatuses consistent with exemplary embodiments relate to a beamforming apparatus that focuses an ultrasound beam, and an ultrasound diagnostic apparatus having the same.

2. Description of the Related Art

Ultrasound diagnostic apparatuses are apparatuses that apply an ultrasound signal from a surface of a subject toward a target region inside a body and obtain a tomogram of soft tissue or an image associated with a blood flow in a noninvasive way using information about the ultrasound signal (e.g., echo signal) reflected from the target region. The ultrasound diagnostic apparatuses have advantages in that ultrasound diagnostic apparatuses are small and inexpensive, can display an image in real time, and have high safety due to no exposure to X-rays, etc., compared to other image diagnostic apparatuses such as X-ray imaging apparatuses, magnetic resonance imaging apparatuses, and nuclear medicine diagnostic apparatuses. The ultrasound diagnostic apparatuses are widely used for diagnosis of cardiac, abdominal, urological, obstetric and gynecological diseases.

Generally, the ultrasound diagnostic apparatuses provide information about a cross section of the interior of the subject in the form of a two-dimensional image using a one-dimensional array transducer. To obtain volume information (three-dimensional information) about the interior of the subject, a method is performed in which a user (e.g., diagnostician, medical doctor) moves the one-dimensional array transducer in a manual or mechanical way (called freehand or mechanical scan). However, this method of obtaining a three-dimensional image based on the manual or mechanical movement of the one-dimensional array transducer is restricted in performance in terms of a temporal or spatial resolution. Therefore, an interest in a technique for obtaining the three-dimensional image using a two-dimensional array transducer is increasing.

### SUMMARY

One or more exemplary embodiments provide a beamforming apparatus capable of transmitting or receiving ultrasound waves to or from some elements that constitute a two-dimensional array transducer and form a specific shape of an aperture and reducing the complexity thereof, an ultrasound probe having the beamforming apparatus, and an ultrasound diagnostic apparatus having the beamforming apparatus.

According to an aspect of an exemplary embodiment, there is provided a beamforming apparatus configured to beamform ultrasound waves transmitted through an ultrasound transducer having a two-dimensional transducer array. The beamforming apparatus includes: a transmitter configured to output transmission pulses configured to drive elements constituting the transducer array; and a transmission switch configured to select at least two elements among the elements to form an aperture such that the transmission pulses drive the elements forming the aperture.

The transducer array may include M×N elements, and a number of the selected elements may be smaller than (M×N)/2, where M and N are natural numbers.

The beamforming apparatus may further include a transmission switch which may be configured to select the elements such that the aperture has a ring shape.

The transmission switch may be configured to select the elements such that a position at which the ring-shaped aperture is formed on the transducer array varies over time.

The transmission switch may be configured to select the elements such that the ring-shaped aperture rotates over time.

The transmission switch may be configured to select the elements such that the aperture has a circular shape or a polygonal shape.

The transmission switch may be configured to select the elements such that a position at which the circular or polygonal aperture is formed on the transducer array varies over time.

The transmission switch may be configured to select the elements such that the circular or polygonal aperture rotates over time.

The transmission switch may be configured to select the elements such that the aperture has a linear shape.

The transmission switch may be configured to select the elements such that a position at which the linear aperture is formed on the transducer array varies over time.

The transmission switch may be configured to select the elements such that the linear aperture rotates over time.

The transmitter may include a transmission beamformer configured to generate transmission signal and add a delay time to the transmission signals and thereby form a transmission signal pattern, and a pulser configured to generate the transmission pulse configured to drive the elements constituting the transducer array according to the transmission signal pattern.

The transmission beamformer and the pulser may be provided plurally, and a number of the transmission beamformers and the pulsers may be smaller than a number of the elements constituting the transducer array.

The beamforming apparatus may further include a transmission/reception switch configured to determine a transmission or reception state of the transducer array.

The transmission switch may include a high-voltage multiplexer.

According to an aspect of another exemplary embodiment, there is provided a beamforming apparatus, which includes: a transmitter configured to output transmission pulses to at least two elements among elements constituting a transducer having a two-dimensional array, the at least two elements forming an aperture,; a receiver configured to focus ultrasound echo signals received by the at least two elements and thereby form a reception beam; and limiters connected to the respective elements constituting the transducer array and configured to protect the receiver from a high voltage occurring when the transmitter is driven.

The transducer array may include M×N elements, and the number of the at least two elements forming the aperture may be smaller than (M×N)/2, where M and N are natural numbers.

The transmitter may include: a transmission beamformer configured to generate transmission signals and add a delay time to the transmission signals to thereby form a transmission signal pattern; and pulsers that are connected to the respective elements constituting the transducer array and are configured to apply the transmission pulses to the respective elements according to the transmission signal pattern.

The receiver may include: pre amplifiers configured to amplify the ultrasound echo signals; a reception switch configured to select the at least two elements forming the aperture; and a reception beamformer configured to focus the ultrasound echo signals and thereby form the reception beam, wherein the reception switch includes a low-voltage multiplexer.

The beamforming apparatus may further include a pulser controller configured to select and drive only some of the pulsers connect to the elements forming the aperture and control the reception switch such that the reception switch selects the elements forming the aperture.

### BRIEF DESCRIPTION OF THE DRAWINGS

The foregoing and other aspects will become more readily apparent upon consideration of the following detailed description of exemplary embodiments taken in conjunction with the accompanying drawings, wherein:

FIG. 1 is a perspective view illustrating an external appearance of an ultrasound diagnostic apparatus according to an exemplary embodiment;

FIG. 2 is a control block diagram of the ultrasound diagnostic apparatus according to an exemplary embodiment;

FIG. 3 is a control block diagram illustrating a detailed configuration of a main body of the ultrasound diagnostic apparatus according to an exemplary embodiment;

FIG. 4 is a control block diagram illustrating a specific configuration of a transmitter according to an exemplary embodiment;

FIG. 5 is a view illustrating a transmit beamforming operation using a one-dimensional array transducer;

FIG. 6 is a view illustrating a delay of a transmission signal applied in the event of transmission beamforming;

FIG. 7 is a view illustrating how to select some of the elements constituting a transducer array to apply transmission signals at the transmitter according to an exemplary embodiment;

FIGS. 8A, 8B, 8C and 8D are views illustrating a shape of an aperture formed by the elements selected by a transmission switch according to an exemplary embodiment;

FIG. 9 is a control block diagram illustrating a specific configuration of a receiver according to an exemplary embodiment;

FIG. 10 is a view illustrating a delay of a reception signal applied in the event of reception beamforming;

FIG. 11 is a view illustrating how to receive electric signals generated from some elements selected from the elements constituting the transducer array at the receiver according to an exemplary embodiment;

FIG. 12 is a block diagram illustrating a configuration of an ultrasound diagnostic apparatus according to another exemplary embodiment;

FIG. 13 is a block diagram illustrating a configuration of an ultrasound probe according to another exemplary embodiment;

FIG. 14 is a block diagram illustrating a configuration of an ultrasound diagnostic apparatus according to yet another exemplary embodiment; and

FIGS. 15 and 16 are views illustrating detailed configurations of a transmitter and receiver of an ultrasound probe according to yet another exemplary embodiment.

### DETAILED DESCRIPTION

Reference will now be made in detail to exemplary embodiments.

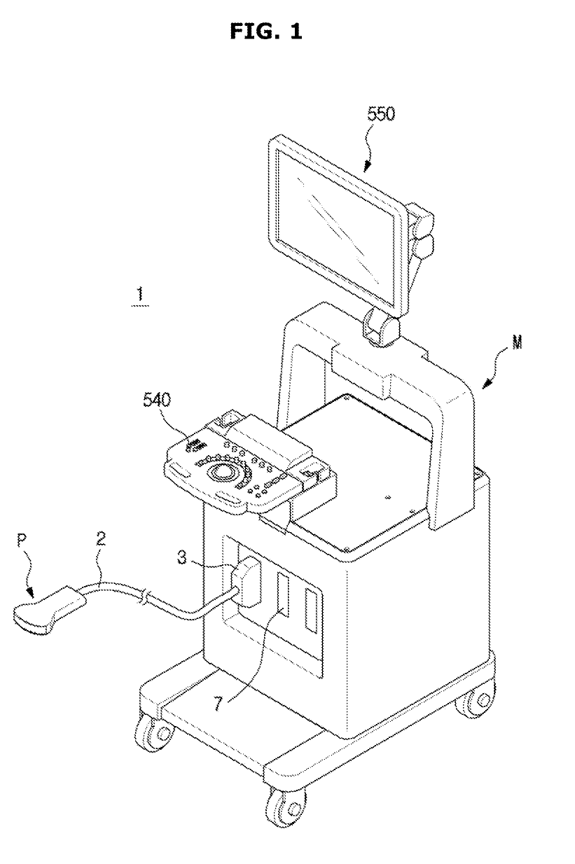

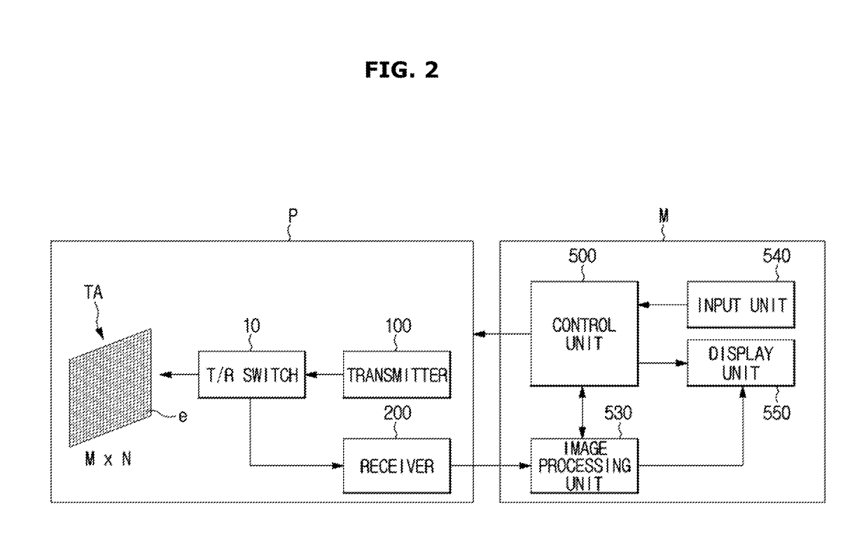

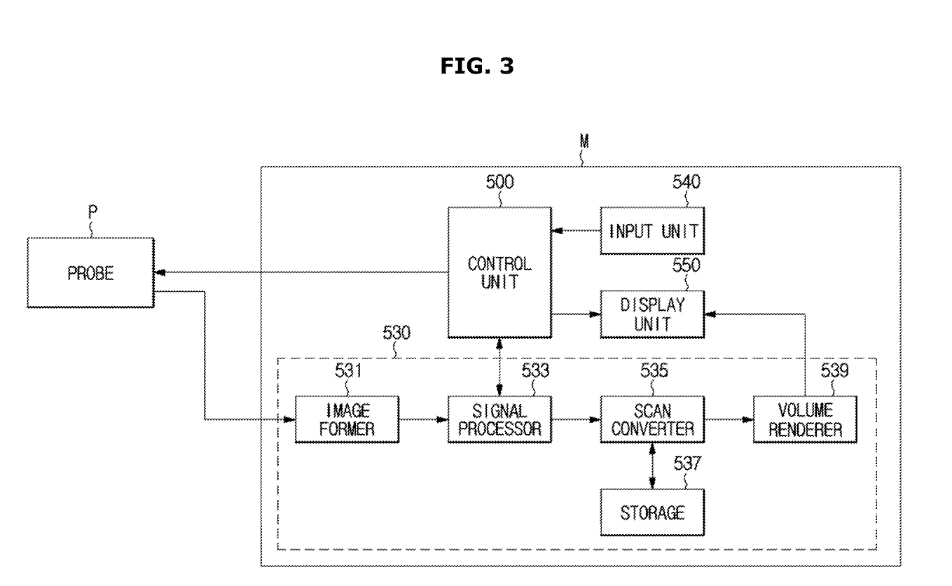

FIG. 1 is a perspective view illustrating an external appearance of an ultrasound diagnostic apparatus according to an exemplary embodiment. FIG. 2 is a control block diagram of the ultrasound diagnostic apparatus according to an exemplary embodiment. FIG. 3 is a control block diagram illustrating a detailed configuration of a main body of the ultrasound diagnostic apparatus according to an exemplary embodiment.

Referring to FIG. 1, an ultrasound diagnostic apparatus 1 includes an ultrasound probe P that transmits ultrasound waves to a subject, receives ultrasound echo waves from the subject, and converts the ultrasound echo waves into electric signals, and a main body M that is equipped with input and display units 540 and 550 connected to the ultrasound probe P and displays an ultrasound image. The ultrasound probe P is connected to the main body M of the ultrasound diagnostic apparatus through a cable 2, and receives various signals required to control the ultrasound probe P or transmits analog or digital signals corresponding to the ultrasound echo signals received by the ultrasound probe P to the main body M. However, the ultrasound probe P is not limited thereto, and may be implemented as a wireless probe that can transceive signals over a network established between the ultrasound probe P and the main body M.

The cable 2 is configured in such a way that one terminal thereof is connected to the ultrasound probe P and the other terminal thereof may be provided with a connector 3 that is removably coupled to a slot 7 of the main body M. The main body M and the ultrasound probe P may transceive control commands or data using the cable 2. For example, when a user inputs information about a focal depth, a size or shape of an aperture, or a steering angle using the input unit 540, the information is transmitted to the ultrasound probe P through the cable 2 so as to be able to be used for transmit and reception beamforming of a transmitter 100 and a receiver 200. Alternatively, if the ultrasound probe P is implemented as the wireless probe as described above, the ultrasound probe P is connected to the main body M over a wireless network instead of the cable 2. Even when the wireless probe is connected to the main body M over the wireless network, the main body M and the ultrasound probe P can transceive the aforementioned control commands or data.

As illustrated in FIG. 2, the main body M may include a control unit 500 (e.g., controller), an image processing unit 530 (e.g., image processor), the input unit 540 (e.g., inputter), and the display unit 550 (e.g., display).

The control unit 500 controls the overall operations of the ultrasound diagnostic apparatus 1. To be specific, the control unit 500 generates control signals for controlling components of the ultrasound diagnostic apparatus 1, for example, the transmitter 100, a transmission/reception (T/R) switch 10, the receiver 200, the image processing unit 530, and the display unit 550 illustrated in FIG. 2, and controls an operation of each of the components. Especially, the control unit 500 calculates delay profiles of multiple ultrasound transducer elements e constituting a two-dimensional ultrasound transducer array TA, and then calculates time delay values depending on distance differences between a focal point of the subject and the multiple ultrasound transducer elements e included in the two-dimensional ultrasound transducer array TA on the basis of the calculated delay profiles. As a result, the control unit 500 controls transmission and reception beamformers such that transmission and reception signals are generated.

Further, the control unit 500 generates a control command of each component of the ultrasound diagnostic apparatus 1 according to an instruction or order of a user which is input through the input unit 540 so as to be able to control the ultrasound diagnostic apparatus 1.

The image processing unit 530 generates a three-dimensional ultrasound image of a target region inside the subject on the basis of the ultrasound signals focused through the receiver 200.

Referring to FIG. 3, the image processing unit 530 may further include an image former 531, a signal processor 533, a scan converter 535, a storage 537, and a volume renderer 539.

The image former 531 generates a coherent two- or three-dimensional image of the target region inside the subject on the basis of the ultrasound signals focused through the receiver 200.

The signal processor 533 converts information about the coherent image formed by the image former 531 into ultrasound image information according to a diagnostic mode such as a B-mode or a D-mode (Doppler mode). For example, when the diagnostic mode is set to the B-mode, the signal processor 533 performs processing such as A/D conversion, and writes out the ultrasound image information for a B-mode image in real time. Further, when an imaging mode is set to the D-mode, the signal processor 533 extracts information about a change in phase from the ultrasound signals, calculates information about a blood flow corresponding to each point of an imaging cross section including a speed, power, and distribution, and writes out the ultrasound image information for a D-mode image in real time.

The scan converter 535 converts the converted ultrasound image information received from the signal processor 533 or the converted ultrasound image information stored in the storage 537 into a video signal for the display unit 550, and transmits the converted result to the volume renderer 539.

The storage 537 provisionally or permanently stores the ultrasound image information converted through the signal processor 533.

The volume renderer 539 performs volume rendering based on a video signal transmitted from the scan converter 535, corrects information about the rendered image to generate a final image, and transmits the generated final image to the display unit 550.

The input unit 540 is provided to enable a user to input the commands for the operations of the ultrasound diagnostic apparatus 1. The user can input or set an instruction to start ultrasound diagnosis, an instruction to select a diagnostic mode such as an amplitude mode (A-mode), a brightness mode (B-mode), a color mode (C-mode), a Doppler mode (D-mode), and a motion mode (M-mode), or region of interest (ROI) setting information including a size and position of an ROI through the input unit 540.

The input unit 540 may include various types of input components, such as a keyboard, a mouse, a trackball, a tablet, or a touchscreen module, through which the user can input data, instructions or commands.

The display unit 550 displays a menu or guidelines required for the ultrasound diagnosis and the ultrasound image obtained during the ultrasound diagnosis. The display unit 550 displays the ultrasound image which the image processing unit 530 generates for the target region inside the subject. The ultrasound image displayed on the display unit 550 may be an A-mode ultrasound image, a B-mode ultrasound image, or a three-dimensional ultrasound image. The display unit 550 may be realized in known various display systems such as a cathode ray tube (CRT), a liquid crystal display (LCD), and so on.

As illustrated in FIG. 2, the ultrasound probe P according to an exemplary embodiment may include an ultrasound transducer array TA, the T/R switch 10, the transmitter 100, and the receiver 200.

The ultrasound transducer array TA is installed on an end of the ultrasound probe P. The ultrasound transducer array TA refers to setting of multiple ultrasound transducer elements e in an array. The ultrasound transducer array TA according to an exemplary embodiment has a two-dimensional array. The ultrasound transducer array TA generates ultrasound waves while vibrating by an applied pulse signal or an applied alternating current. The generated ultrasound waves are transmitted to the target region inside the subject. In this case, the ultrasound waves generated by the ultrasound transducer array TA may be transmitted by focusing on multiple target regions inside the subject. In other words, the generated ultrasound waves may be transmitted to the multiple target regions by multi-focusing.

The ultrasound waves generated by the ultrasound transducer array TA are reflected from the target region inside the subject, and then return to the ultrasound transducer array TA. The ultrasound transducer array TA receives the ultrasound echo waves reflected back from the target region. When the ultrasound echo waves arrive, the ultrasound transducer array TA vibrates at a frequency corresponding to a frequency of the ultrasound echo waves, and outputs an alternating current of a frequency corresponding to the vibration frequency. Thus, the ultrasound transducer array TA can convert the received ultrasound echo waves into predetermined electric signals.

Since the elements e receive the ultrasound echo waves to output the electric signals, the ultrasound transducer array TA can output the electric signals of multiple channels. The number of channels may be set to the same number as the ultrasound transducer elements e constituting the ultrasound transducer array TA. However, as in the exemplary embodiment, if the ultrasound transducer array TA is formed as the two-dimensional array, the number of channels is considerably increased compared to when the ultrasound transducer array TA is formed as the one-dimensional array. An increase in the number of channels makes a system complicated, increases costs required to implement the system, and makes it difficult to realize a compact apparatus. For this reason, an exemplary embodiment provides the ultrasound diagnostic apparatus 1 capable of obtaining a three-dimensional volume image using a transducer having a two-dimensional array without increasing the number of channels. Details will be described below.

Each of the ultrasound transducer elements e may include a piezoelectric vibrator or a thin film. When an alternating current is applied to the piezoelectric vibrator or the thin film from a power source, the piezoelectric vibrator or the thin film vibrates at a predetermined frequency according to the applied alternating current, and generates a predetermined frequency of ultrasound wave according to the vibration frequency. In contrast, when a predetermined frequency of ultrasound echo waves arrive at the piezoelectric vibrator or the thin film, the piezoelectric vibrator or the thin film vibrates according to the ultrasound echo wave, and outputs an alternating current of a frequency corresponding to the vibration frequency.

The ultrasound transducer may be implemented as any one of a magnetostrictive ultrasonic transducer using a magnetostrictive effect of a magnet, a piezoelectric ultrasonic transducer using a piezoelectric effect of a piezoelectric material, and a capacitive micro-machined ultrasonic transducer (cMUT) transceiving ultrasonic waves using vibration of several hundreds or thousands of micro-machined thin films. In addition, other types of transducers capable of generating an ultrasonic wave according to an electric signal or vice versa may be implemented as types of the ultrasound transducer.

The transmitter 100 applies transmission pulses to the transducer array TA so as to cause the transducer array TA to transmit ultrasound signals to the target region inside the subject.

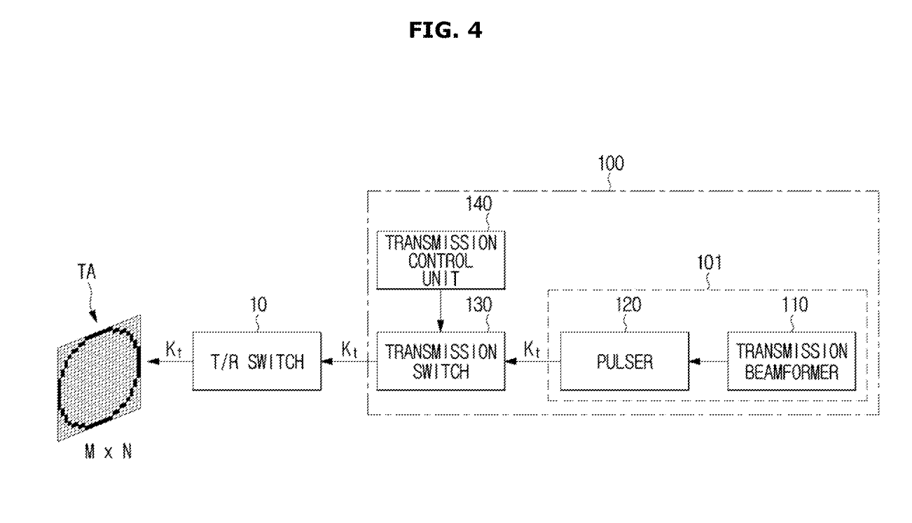

FIG. 4 is a control block diagram illustrating a specific configuration of a transmitter according to an exemplary embodiment. As illustrated in FIG. 4, the transmitter 100 includes: a transmission beamforming apparatus 101 having a transmission beamformer 110 and a pulser 120; a transmission switch 130; and a transmission control unit 140 (e.g., transmission controller).

The transmission beamformer 110 forms a transmission signal pattern according to the control signal of the control unit 500 of the main body M, and outputs the formed transmission signal pattern to the pulser 120. The transmission beamformer 110 forms the transmission signal pattern on the basis of the time delay value which the control unit 500 calculates for each of the ultrasound transducer elements e constituting the two-dimensional ultrasound transducer array TA, and transmits the formed transmission signal pattern to the pulser 120.

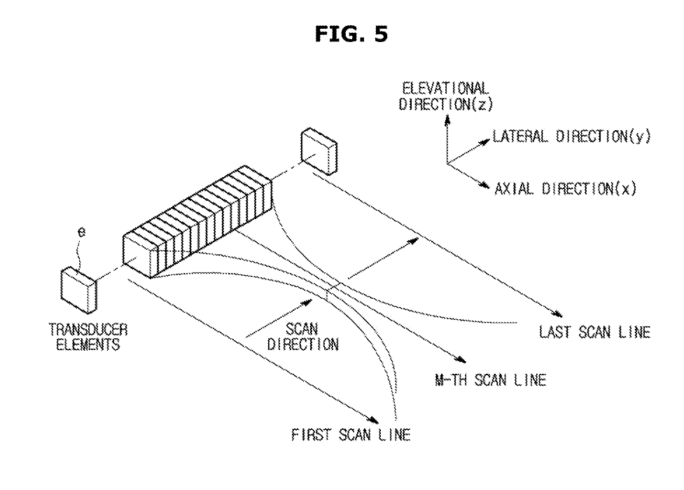

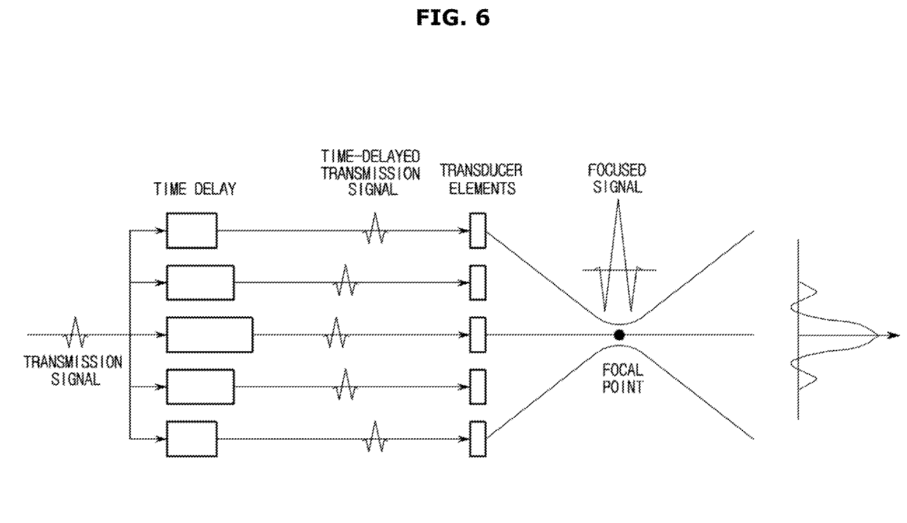

The transmission beamforming will be described in greater detail with reference to FIGS. 5 and 6. FIG. 5 is a view illustrating a transmission beamforming operation using a one-dimensional array transducer, and FIG. 6 is a view illustrating a delay of a transmission signal applied in the event of transmission beamforming.

In an exemplary embodiment, in spite of using the two-dimensional array transducer, the transmission beamforming will be described taking the one-dimensional array transducer by way of example for the convenience of description. As illustrated in FIG. 5, a three-dimensional space in which ultrasound imaging is performed can be defined by a z axis corresponding to an elevational direction, a y axis corresponding to a lateral direction, and an x axis corresponding to an axial direction.

A spatial resolution of the two-dimensional ultrasound image can be determined by resolutions in the axial and lateral directions. The resolution in the axial direction refers to the capability of distinguishing two objects arranged along an axis of an ultrasound beam, and the resolution in the lateral direction refers to the capability of distinguishing two objects arranged perpendicular to the axis of the ultrasound beam.

The resolution in the axial direction is determined by a pulse width of a transmitted ultrasound signal. The narrower the pulse width of the high-frequency ultrasound signal, the higher the resolution in the axial direction. Resolutions in the lateral and elevational directions are determined by a width of the ultrasound beam. The narrower the ultrasound beam, the higher the resolution in the lateral direction.

Thus, in order to improve the resolution of the ultrasound image, and particularly the resolution in the lateral direction, the ultrasound signals transmitted from the multiple transducer elements e are focused on a focal point on scan lines, and thereby the ultrasound beam whose width is narrow can be formed, which is called transmission beamforming.

A one-dimensional (1D) array transducer is made up of multiple transducer elements e arranged in one dimension. In order to obtain a two-dimensional ultrasound cross-sectional image, multiple scan lines are required, and the beamforming can be performed on the aforementioned focal point from a first scan line to the final scan line.

When the ultrasound signals are transmitted with respect to all the scan lines and ultrasound echo signals reflected back from the target region inside the subject are received, a two-dimensional ultrasound cross-sectional image on an xy plane can be obtained.

To focus the ultrasound beam on one point, the ultrasound signals transmitted from the multiple transducer elements e should be able to reach one focal point. As illustrated in FIG. 6, since distances from the elements e to the focal point are different from each other, the ultrasound signals transmitted from the respective elements e are configured to be able to reach the same focal point at the same time by giving a proper time delay to the transmitted ultrasound signals.

Referring to FIG. 6, when the ultrasound signals are simultaneously transmitted from all the elements e toward the focal point, the ultrasound signal transmitted from the element nearest the focal point arrives at the focal point first. As the element becomes distant from the focal point, an arrival time is delayed.

Thus, as illustrated in FIG. 6, in view of the time delay in giving a transmission signal to the element, the transmission signal may be given to the element nearest the focal point last. As the element becomes distant from the focal point, the transmission signal may be gradually given early. Here, the transmission signal refers to an electric signal that is converted into the ultrasound signal at the element.

The transmission beamforming apparatus 101 including the transmission beamformer 110 and the pulser 120 may be provided as many as a number corresponding to the number of elements e constituting the transducer array TA. According to this configuration, the number of channels of the apparatus is increased by the number of elements e, and the ultrasound diagnostic apparatus 1 is increased in complexity and is rarely realized.

Therefore, an exemplary embodiment provides the ultrasound probe P and the ultrasound diagnostic apparatus 1, both of which are capable of obtaining the three-dimensional volume image with only as many channels as the ultrasound diagnostic apparatus 1 using the one-dimensional array transducer has, despite using the two-dimensional array transducer. The transmission beamforming apparatuses 101 according to an exemplary embodiment are provided by Kt that is a number smaller than M×N that is the number of the elements e constituting the two-dimensional array transducer, where Kt, M, and N are the natural numbers. For example, when the two-dimensional array transducer has an M×N array, the Kt transmission beamforming apparatuses 101 may be provided as many as an N number. In detail, the number of the Kt transmission beamforming apparatuses 101 may be smaller than ½, ¼, ⅛, or 1/16 of M×N that is the number of the elements constituting the two-dimensional array transducer.

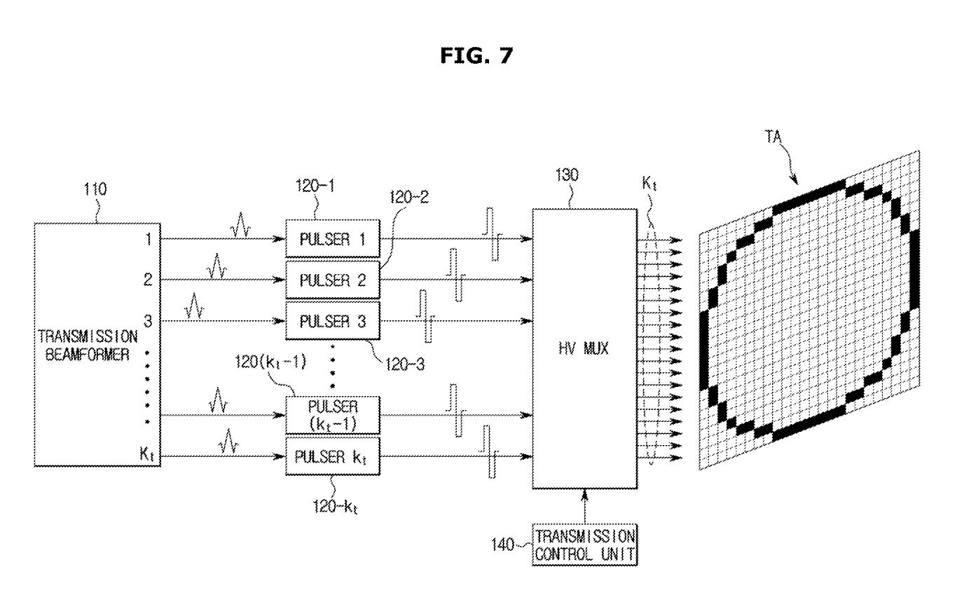

Thus, the transmission beamforming apparatuses 101 output Kt transmission signals, and the Kt transmission signals are applied to Kt elements e of the M×N elements e. The transmission switch 130 selects the Kt elements, to which the Kt transmission signals output from the transmission beamforming apparatuses 101 are to be applied, from the M×N elements e, and performs switching such that the selected elements e form a predetermined shape of an aperture. The transmission control unit 140 controls the transmission switch 130 such that the elements e selected by the transmission switch 130 form the predetermined shape of aperture AP. FIG. 7 is a view illustrating how to select some of the elements e constituting the transducer array TA to apply transmission signals at the transmitter 100 according to an exemplary embodiment. How to select the elements e at the transmission switch 130 will be described in greater detail with reference to FIG. 7.

As illustrated in FIG. 7, the transmitter 100 includes the transmission switch 130 selectively activating the transducer elements e between the pulser 120 and the transducer array TA. As illustrated in FIG. 7, a high-voltage multiplexer (HV MUX) may be used as the transmission switch 130, but the HV MUX is merely an example. Any other type of component capable of selectively activating the elements e may be included in or implemented as the transmission switch 130.

As illustrated in FIG. 7, the transmission beamformer 110 outputs Kt transmission signals subjected to transmission beamforming. Kt pulsers 120 (e.g., 120-1, 120-2, 120-3, 120 (Kt−1), 120 (Kt)) are provided to be able to receive Kt time-delayed transmission signals that are output from the transmission beamformer 110 and are subjected to time delays. The respective pulsers 120 receive the Kt time-delayed transmission signals to generate Kt high-voltage transmission pulses, and apply the Kt high-voltage transmission pulses to the Kt elements e selected from the M×N elements e by the transmission switch 130.

The transmission switch 130 selects the Kt elements e from the M×N elements e such that the Kt transmission pulses output from the pulsers 120 are transmitted to the selected elements e. The transmission control unit 140 controls the switching of the transmission switch 130 such that the Kt elements e selected by the transmission switch 130 can form a predetermined shape of aperture AP. For example, as illustrated in FIG. 7, the transmission control unit 140 controls the transmission switch 130 such that the aperture AP formed of the elements e selected by the transmission switch 130 can have a ring shape.

The shape of the aperture AP is selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to the transmission control unit 140 of the ultrasound probe P. The transmission control unit 140 controls an operation of the transmission switch 130 on the basis of the information transmitted from the control unit 500 of the main body M, thereby forming the aperture AP having the ring shape as illustrated in FIG. 7. Without the control of the control unit 500 of the main body M, the transmission control unit 140 may directly control the transmission switch 130 in response to input of a command to select the shape of the aperture AP.

Although there is no limitation on a shape in which the aperture AP can be realized, the exemplary embodiment may control the transmission switch 130 such that the aperture AP has a circular or oval shape, or the ring shape as illustrated in FIG. 7, so as to be able to maintain isotropy of a resolution in a space.

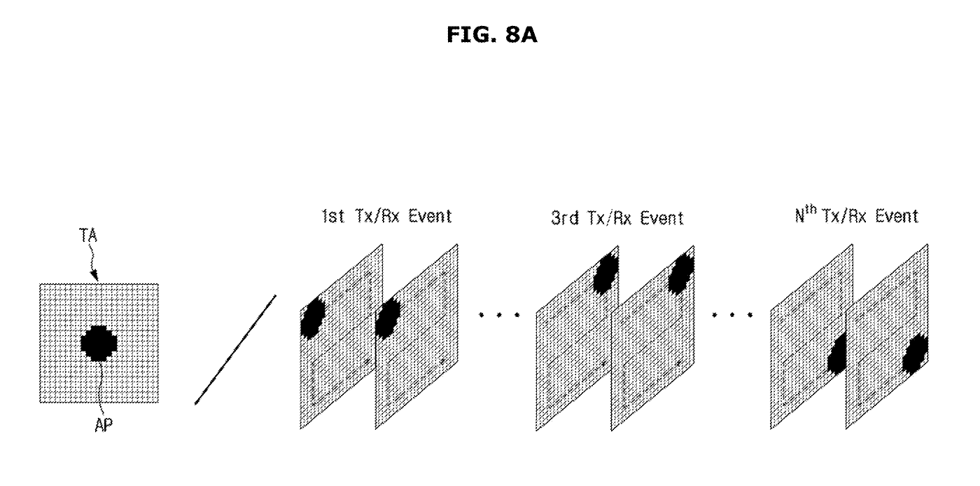

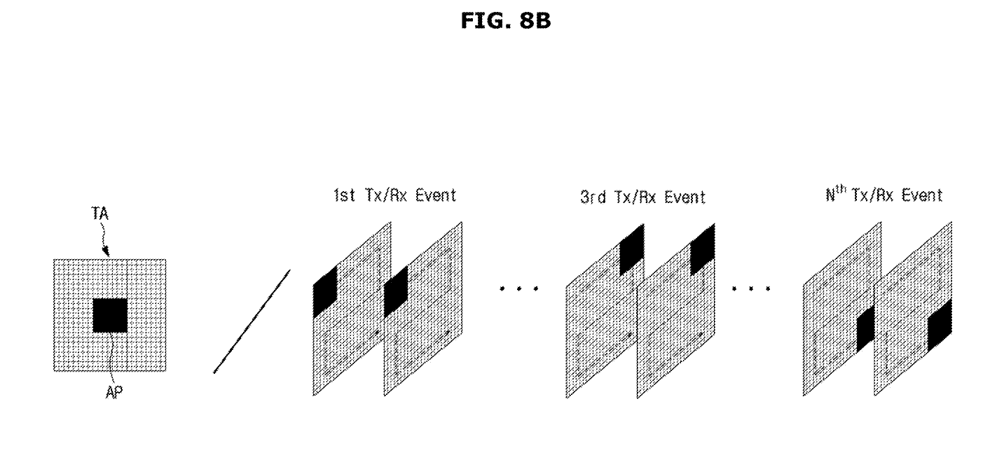

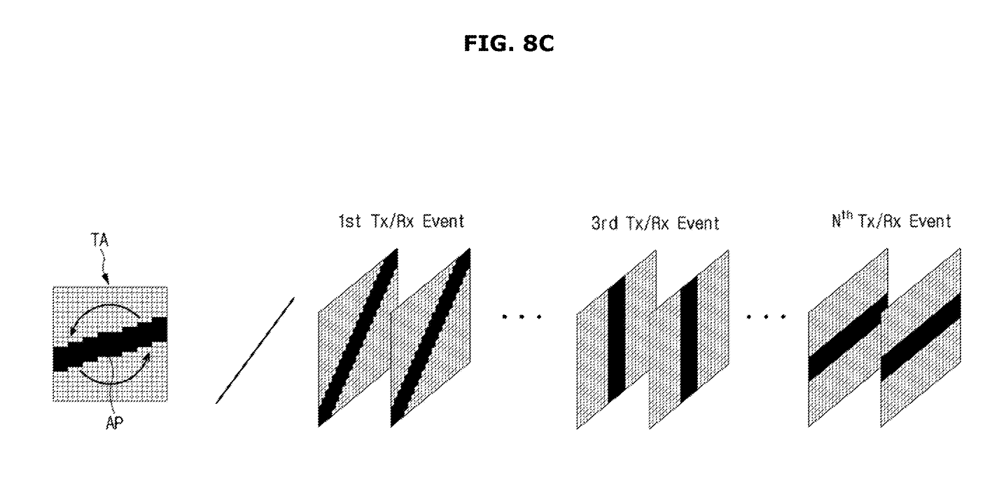

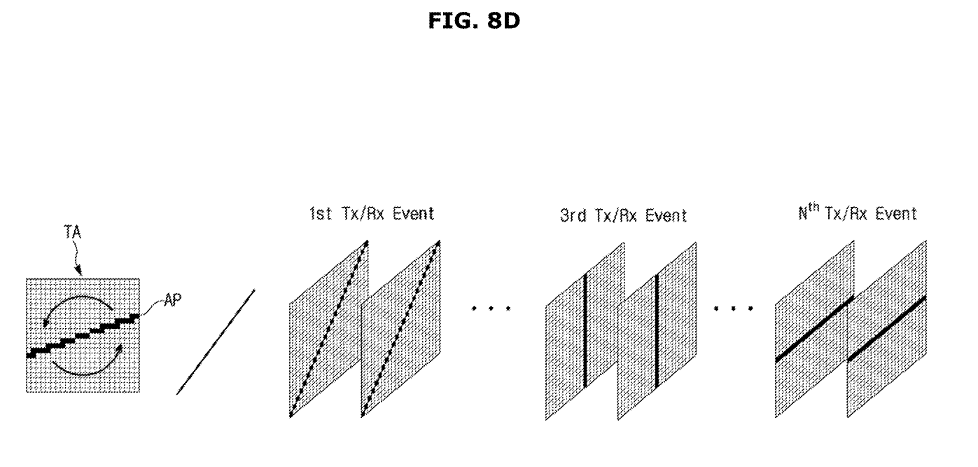

FIGS. 8A to 8D are views illustrating the shape of the aperture AP formed by the elements e selected by the transmission switch 130 according to an exemplary embodiment. As illustrated in FIG. 8A, the aperture AP may be realized in a circular shape. Further, the aperture AP may be realized in an oval shape. The circular aperture AP is not restricted in size and formed position. As illustrated in FIG. 8A, the circular aperture AP may be formed at another position after a transmission or reception event is completed once. In other words, the transmission or reception event may be performed while the aperture AP moves over time. A moving path or direction of the aperture AP illustrated in the figure is merely an example. The aperture AP may randomly move without an established rule, and be subjected to a variation in size or shape while moving.

Further, as illustrated in FIG. 8B, the aperture AP may be realized in a polygonal shape including a quadrangular shape. The polygonal aperture AP is not restricted in size and formed position. As illustrated in FIG. 8B, the polygonal aperture AP may be formed at another position after the transmission or reception event is completed once. In other words, the transmission or reception event may be performed while the aperture AP moves over time. A moving path or direction of the aperture AP illustrated in the figure is merely an example. The aperture AP may randomly move without an established rule, and may be subjected to a variation in size or shape while moving. Further, as illustrated in FIG. 8C, the polygonal aperture AP may have a shape running through the transducer array TA in a vertical or horizontal direction. The transmission or reception event may be performed while the polygonal aperture AP rotates over time. The polygonal aperture AP is not restricted in size, form, and formed position, and may be subjected to a variation in size or form while rotating. Further, the aperture AP running through the transducer array TA in a vertical or horizontal direction in an over shape other than the polygonal shape may be formed, and the transmission or reception event may be performed while the oval aperture AP rotates over time.

Further, as illustrated in FIG. 8D, the aperture AP may be realized in a linear shape. For example, the aperture AP may be realized in a one-dimensional array shape. Any linear shape thicker than the linear one-dimensional array shape may be included in the aforementioned polygonal shape. The aperture AP realized in the linear shape may have a shape running through the transducer array TA in a vertical or horizontal direction. As illustrated in FIG. 8D, the transmission or reception event may be performed while the linear aperture AP rotates over time.

The transmission switch 130 may select the elements e under the control of the transmission control unit 140, and form the aperture AP in the shape illustrated in FIGS. 8A to 8D to perform the transmission or reception event. The ultrasound diagnostic apparatus 1 may obtain a three-dimensional volume image from ultrasound echo signals received with the aforementioned aperture AP. The shapes of the aperture AP which are illustrated in FIGS. 8A to 8D are merely examples, and the aperture AP may be formed in other shapes.

The receiver of FIG. 2 performs predetermined processing on the ultrasound echo signals received from the transducer array TA, and performs reception beamforming.

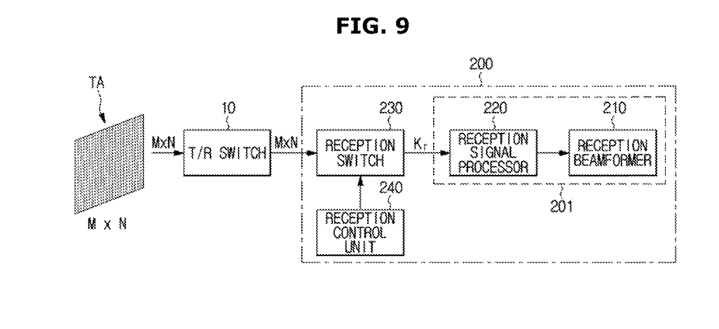

FIG. 9 is a control block diagram illustrating a specific configuration of the receiver according to an exemplary embodiment. As illustrated in FIG. 9, the receiver 200 includes a reception switch 230, a reception control unit 240 (e.g., reception controller) that controls switching of the reception switch 230, and a reception beamforming unit 201 (e.g., reception beamformer) that includes a reception signal processor 220 and a reception beamformer 210.

First, the reception beamforming will be described along with the reception beamforming unit 201, and then the reception switch 230 and the reception control unit 240 will be described.

The ultrasound echo signals reflected back from the focal point are input to the transducer array TA, and the transducer array TA converts the input ultrasound echo signals into analog electric signals.

The electric signals converted by the transducer array TA are input to the reception signal processor 220. The reception signal processor 220 may amplify the electric signals into which the ultrasound echo signals are converted before signal processing or time delay processing, and adjust a gain or compensate for attenuation according to a depth. To be more specific, the reception signal processor 220 may include a low noise amplifier (LNA) that reduces noise of the electric signals input from the ultrasound transducer array TA, and a variable gain amplifier (VGA) that controls a gain value according to an input signal. The VGA may include, but is not limited to including, a time gain compensation (TGC) amplifier that compensates for a gain according to a distance from a focal point.

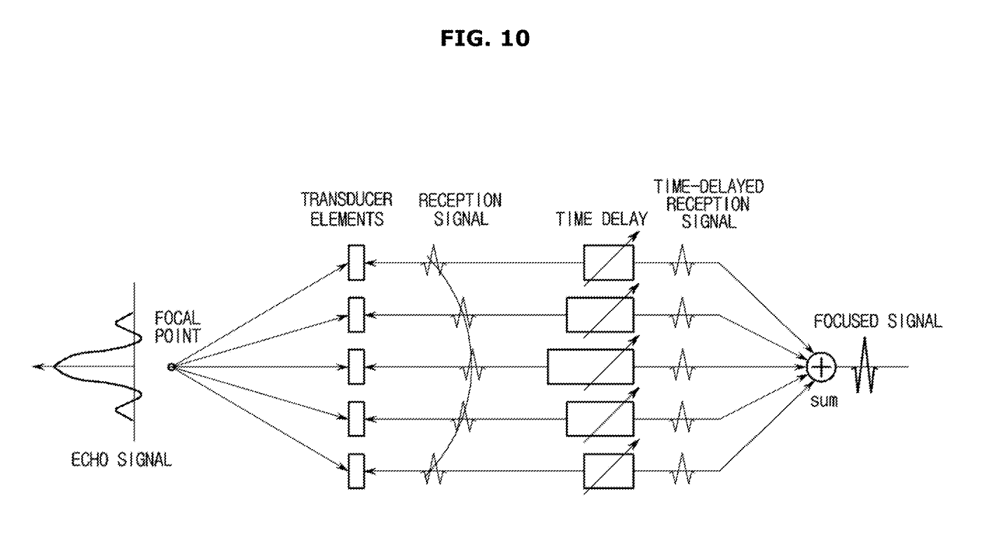

The reception beamformer 210 performs beamforming on the electric signals input from the reception signal processor 220. The reception beamformer 210 strengthens signal intensity by superpositioning the electric signals input from the reception signal processor 220. The reception beamforming will be described in detail with reference to FIG. 10. FIG. 10 is a view illustrating a delay of a reception signal applied in the event of the reception beamforming. As described in FIG. 6 above, when the ultrasound waves of the same phase arrive at the focal point by performing the transmission beamforming, the ultrasound echo signals are generated from the focal point, and then return to the transducer array TA.

Similar to when the ultrasound waves are transmitted to the focal point, when the ultrasound echo signals are received from the focal point, distances from the transducer elements e to the focal point are different from each other. As a result, the ultrasound echo signals are different in arrival time from each other. In detail, the ultrasound echo signal arrives at the element nearest the focal point first, and arrives at the element farthest from the focal point last.

Since magnitudes of the ultrasound echo signals are very small, it is difficult to obtain information only from the single signal received by each element e. Similar to the transmission beamforming, the reception beamforming is configured to give a proper time delay to the signals that arrive at the respective elements e with a time lag, and to sum up the signals at the same time, thereby improving a signal-to-noise ratio.

The signals beamformed by the reception beamformer 210 are converted into digital signals by an AD converter, and are transmitted to the image processing unit 530 of the main body M. When the AD converter is provided for the main body M, the analog signals beamformed by the reception beamformer 210 may be transmitted to the main body M, and may be converted into the digital signals at the main body M. Alternatively, the reception beamformer 210 may be a digital beamformer. In this case, the digital beamformer may include a storage capable of sampling and storing an analog signal, a sampling period control unit capable of controlling a sampling period, an amplifier capable of adjusting a size of a sample, an anti-aliasing low pass filter for preventing aliasing before the sampling, a band-pass filter capable of selecting a desired frequency band, an interpolation filter capable of increasing a sampling rate in the event of beamforming, and a high-pass filter capable of removing a direct current (DC) component or a low-frequency band signal.

In an exemplary embodiment, it has been described that the signals travel through the reception beamformer 210 once. However, the signals may travel through the reception beamformer 210 two or more times so as to further reduce the number of channels. The signals are output from the reception beamformer 210 after the input signals are summed up, and thus the number of signals output from the reception beamformer 210 is smaller than the number of input signals. Therefore, the number of channels can be reduced by causing the signals to travel through the reception beamformer 210 several times.

As illustrated in FIG. 9, the receiver according to an exemplary embodiment includes the reception beamforming unit 201 that includes the reception signal processor 220 and the reception beamformer 210, and the reception switch 230 that selects the elements e such that the electric signals generated from the elements e selected from the transducer elements e are input to the reception beamforming unit 201. Similar to the transmission switch 130, the reception switch 230 according to an exemplary embodiment selects the elements e, the number of which is Kr smaller than M×N that is the number of the elements e constituting the two-dimensional array transducer, where Kr, M, and N are the natural numbers. Thereby, the ultrasound diagnostic apparatus 1 according to an exemplary embodiment can obtain a three-dimensional volume image with only as many channels as the ultrasound diagnostic apparatus 1 using the one-dimensional array transducer has, despite using the two-dimensional array transducer. The reception switch 230 selects the Kr elements from the transducer elements e, receives the ultrasound echo signals from the selected elements e, and transmits generated electric signals to the reception beamforming unit 201. The reception control unit 240 controls an operation of the reception switch 230 such that the elements e selected by the reception switch 230 form a predetermined shape of aperture AP.

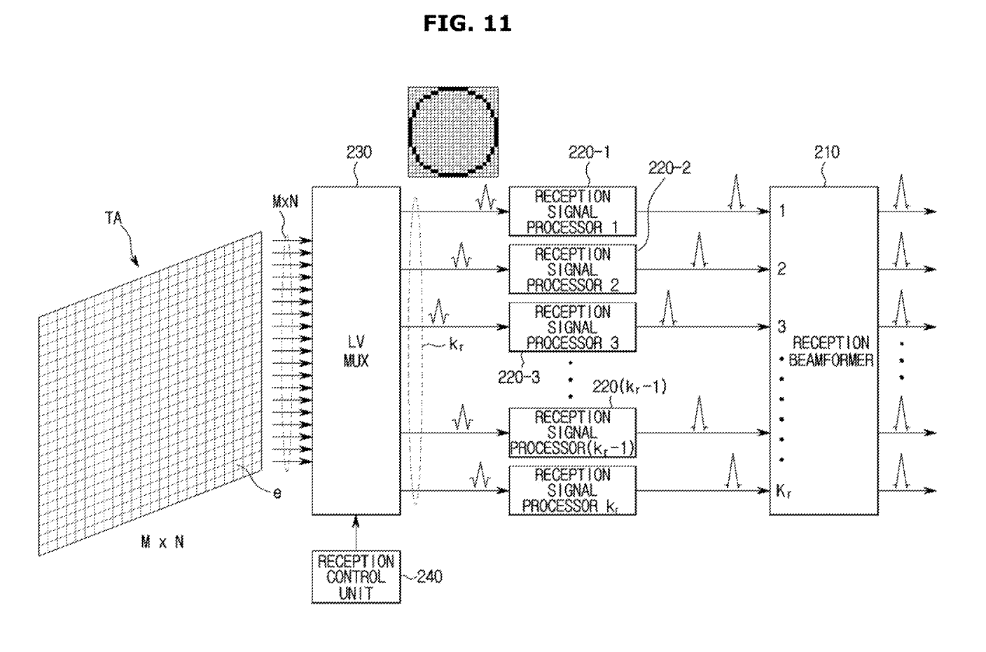

FIG. 11 is a view illustrating how to receive electric signals generated from some elements selected from the elements constituting the transducer array TA at the receiver 200 according to an exemplary embodiment. How the elements are selected by the reception switch 230 will be described in greater detail with reference to FIG. 11.

As illustrated in FIG. 11, the receiver 200 includes the reception switch 230 selectively activating the transducer elements e between the reception signal processor 220 and the transducer array TA. As illustrated in FIG. 11, a low-voltage multiplexer (LV MUX) may be used as the reception switch 230, but the LV MUX is merely an example. Any other type of component capable of selectively activating the elements e may be included in the reception switch 230.

As illustrated in FIG. 11, the reception switch 230 selects the Kr number of elements e from the M×N number of elements e under the control of the reception control unit 240 such that the electric signals generated from the selected elements e are input to the reception signal processor 220. The reception control unit 240 controls the switching of the reception switch 230 such that the Kr elements e selected by the reception switch 230 can form a predetermined shape of aperture AP. For example, the reception control unit 240 may control the reception switch 230 such that the aperture AP formed of the elements e selected by the reception switch 230 can have a ring shape, similar to the transmission aperture AP illustrated in FIG. 1. The Kr elements e selected by the reception switch 230 may be equal to the Kt elements e selected by the transmission switch 130.

The shape of the aperture AP is selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to the reception control unit 240 of the ultrasound probe P. The reception control unit 240 controls an operation of the reception switch 230 on the basis of the information transmitted from the control unit 500 of the main body M, thereby determining the shape of the aperture AP. Without the control of the control unit 500 of the main body M, the reception control unit 240 may directly control the reception switch 230 in response to input of a command to select the shape of the aperture AP.

Although there is no limitation on a shape in which the aperture AP can be realized, an exemplary embodiment may control the reception switch 230 such that the aperture AP has a circular or oval shape, or a ring shape, so as to be able to maintain isotropy of a resolution in a space. The description of the shape of the aperture AP will be omitted because it may be identical to the description made with reference to FIG. 8.

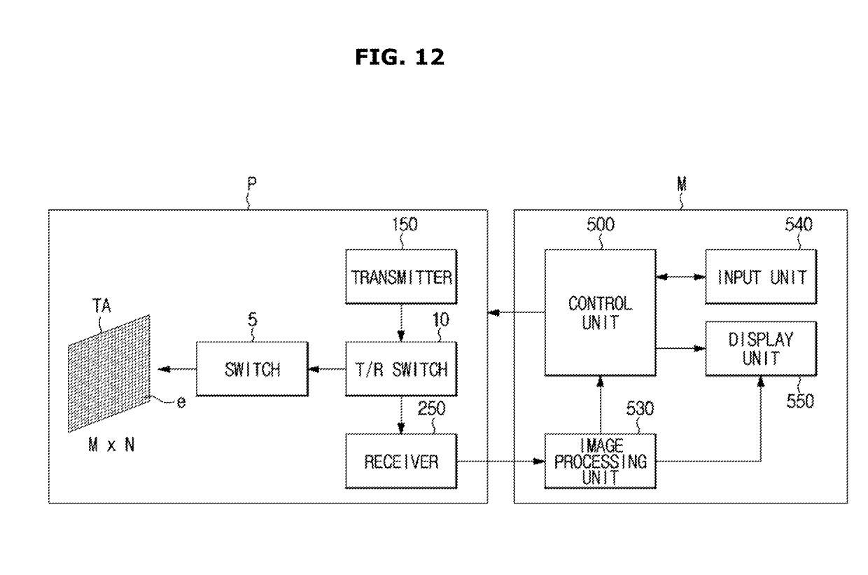

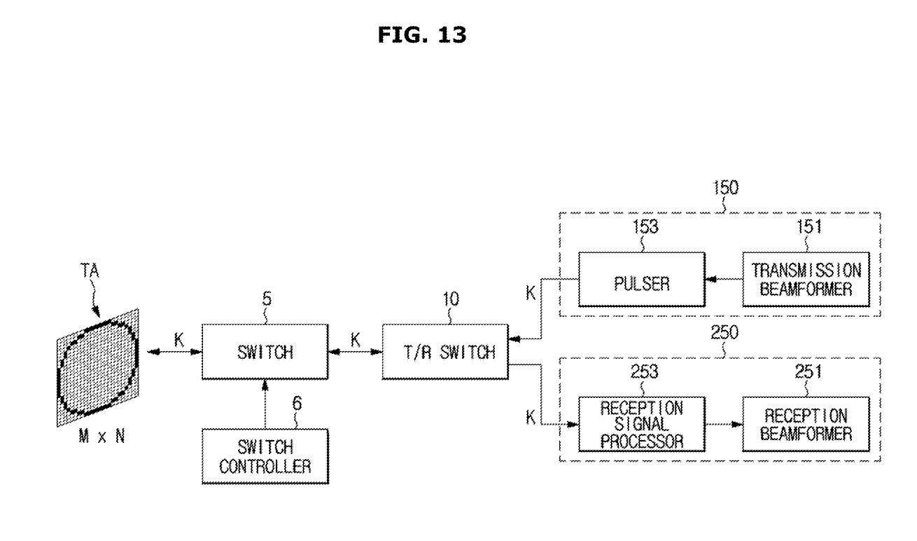

FIG. 12 is a block diagram illustrating a configuration of an ultrasound diagnostic apparatus according to another exemplary embodiment. FIG. 13 is a block diagram illustrating a configuration of an ultrasound probe according to another exemplary embodiment. A main body M of the ultrasound diagnostic apparatus according to the other exemplary embodiment will not be described because the main body M of the ultrasound diagnostic apparatus according to the other exemplary embodiment may have the same configuration as the main body M of the aforementioned exemplary embodiment.

The ultrasound probe P according to the other exemplary embodiment includes a transducer array TA, a switch 5, a T/R switch 10, a transmitter 150, and a receiver 250.

In the present exemplary embodiment, the single switch 5 selects elements when ultrasound waves are transmitted or received, unlike the aforementioned exemplary embodiment in which the transmission switch 130 selects the elements e when the ultrasound waves are transmitted and the reception switch 230 selects the elements e when the ultrasound waves are received are respectively provided for the transmitter 150 and the receiver 250.

When a pulser 153 of the transmitter 150 is connected to the switch 5 by the T/R switch 10, transmission pulses are applied to the elements selected by the switch 5.

To be more specific, a transmission beamformer 151 forms a transmission signal pattern according to a control signal of a control unit 500 of the main body M, and outputs the formed transmission signal pattern to the pulser 153. The transmission beamformer 151 forms the transmission signal pattern on the basis of time delay values that are calculated with respect to the ultrasound transducer elements e constituting the two-dimensional ultrasound transducer array TA by the control unit 500, and transmits the formed transmission signal pattern to the pulser 153.

The transmission beamformer 151 outputs a transmission signal pattern constituting K transmission signals subjected to the transmission beamforming, where K is a natural number. K pulsers 153 are provided to be able to receive K time-delayed transmission signals output from the transmission beamformer 151. The pulsers 153 receive the K time-delayed transmission signals to generate K transmission pulses, and apply the generated transmission pulses to K elements that are selected from the M×N elements e by the switch 5.

The switch 5 selects the K elements e from the M×N elements e such that the K transmission pulses output from the pulsers 153 are transmitted to the K selected elements e. The switch controller 6 controls switching of the switch 5 such that the K elements e selected by the switch 5 can form a predetermined shape of aperture AP. For example, the switch controller 6 may control the switch 5 such that the aperture AP formed by the elements e selected by the switch 5 can have a ring shape.

The shape of the aperture AP may be selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to a switch controller 6 of the ultrasound probe P. The switch controller 6 controls an operation of the switch 5 on the basis of the information transmitted from the control unit 500 of the main body M, thereby making it possible to determine the shape of the aperture AP. Without the control of the control unit 500 of the main body M, the switch controller 6 may directly control the switch 5 in response to input of a command to select the shape of the aperture AP. Although there is no limitation on a shape in which the aperture AP can be realized, an exemplary embodiment may control the switch 5 such that the aperture AP has a circular or oval shape, or the ring shape, as illustrated in FIG. 7, so as to be able to maintain isotropy of a resolution in a space like the aforementioned exemplary embodiment.

When the reception signal processor 253 of the receiver 250 is connected to the switch 5 by the T/R switch 10, electric signals generated from the elements selected by the switch 5 are input to the reception signal processor 253.

To be more specific, similar to the switch 5 in a transmitted state, the switch 5 in a received state selects the elements e, the number of which is K smaller than M×N that is the number of elements e constituting the two-dimensional array transducer. The switch 5 selects the K elements e from the M×N elements under the control of the switch controller 6 such that the electric signals generated from the selected elements e are input to the reception signal processor 253. The switch controller 6 controls the switching of the switch 5 such that the K elements e selected by the switch 5 can form a predetermined shape of aperture AP. For example, the switch controller 6 may control the switch 5 such that the aperture AP formed by the elements selected by the switch 5 can have the ring shape like the aperture AP in the transmitted state. The K elements selected by the switch 5 may be equal to the K elements selected by the switch 5 in the transmitted state.

The shape of the aperture AP is selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to a switch controller 6 of the ultrasound probe P. The switch controller 6 controls an operation of the switch 5 on the basis of the information transmitted from the control unit 500 of the main body M, thereby determining the shape of the aperture AP. Without the control of the control unit 500 of the main body M, the switch controller 6 may directly control the switch 5 in response to input of a command to select the shape of the aperture AP. Although there is no limitation on a shape in which the aperture AP can be realized as in the transmitted state, the switch controller 6 may control the switch 5 such that the aperture AP has a circular or oval shape, or a ring shape, so as to be able to maintain isotropy of a resolution in a space.

The electric signals converted by the transducer array TA are input to the reception signal processor 253. The reception signal processor 253 may amplify the electric signals into which ultrasound echo signals are converted before signal processing or time delay processing, and adjust a gain or compensate for attenuation according to a depth. To be more specific, the reception signal processor 253 may include a low noise amplifier (LNA) that reduces noise of the electric signals input from the ultrasound transducer array TA, and a variable gain amplifier (VGA) that controls a gain value according to an input signal. The VGA may include, but is not limited to including, a time gain compensation (TGC) amplifier that compensates for a gain according to a distance from a focal point.

The reception beamformer 251 performs beamforming on the electric signals input from the reception signal processor 253. The reception beamformer 251 strengthens signal intensity by superpositioning the electric signals input from the reception signal processor 253.

When the ultrasound waves of the same phase arrive at the focal point by performing the transmission beamforming, the ultrasound echo signals are generated from the focal point, and then return to the transducer array TA. Similar to when the ultrasound waves are transmitted to the focal point, when the ultrasound echo signals are received from the focal point, distances from the transducer elements e to the focal point are different from each other. As a result, the ultrasound echo signals are different in arrival time from each other. In detail, the ultrasound echo signal arrives at the element nearest the focal point first, and arrives at the element farthest from the focal point last.

Since magnitudes of the ultrasound echo signals are very small, it is difficult to obtain information only from the single signal received by each element e. Similar to the transmission beamforming, the reception beamforming is configured to give a proper time delay to the signals that arrive at the respective elements e with a time lag, and to sum up the signals at the same time, thereby improving a signal-to-noise ratio.

The signals beamformed by the reception beamformer 251 are converted into digital signals by an AD converter, and are transmitted to the image processing unit 530 of the main body M. When the AD converter is provided for the main body M, the analog signals beamformed by the reception beamformer 251 may be transmitted to the main body M, and may be converted into the digital signals at the main body M. Alternatively, the reception beamformer 251 may be a digital beamformer. In this case, the digital beamformer may include a storage capable of sampling and storing an analog signal, a sampling period control unit capable of controlling a sampling period, an amplifier capable of adjusting a size of a sample, an anti-aliasing low pass filter for preventing aliasing before the sampling, a band-pass filter capable of selecting a desired frequency band, an interpolation filter capable of increasing a sampling rate in the event of beamforming, and a high-pass filter capable of removing a direct current (DC) component or a low-frequency band signal. In an exemplary embodiment, it has been described that the signals travel through the reception beamformer 251 once. However, the signals may travel through the reception beamformer 251 two or more times so as to further reduce the number of channels. The signals are output from the reception beamformer 251 after the input signals are summed up, and thus the number of signals output from the reception beamformer 251 is smaller than the number of input signals. Therefore, the number of channels can be reduced by causing the signals to travel through the reception beamformer 251 several times.

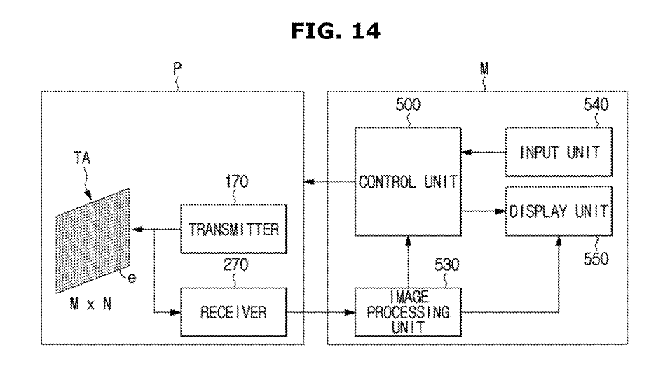

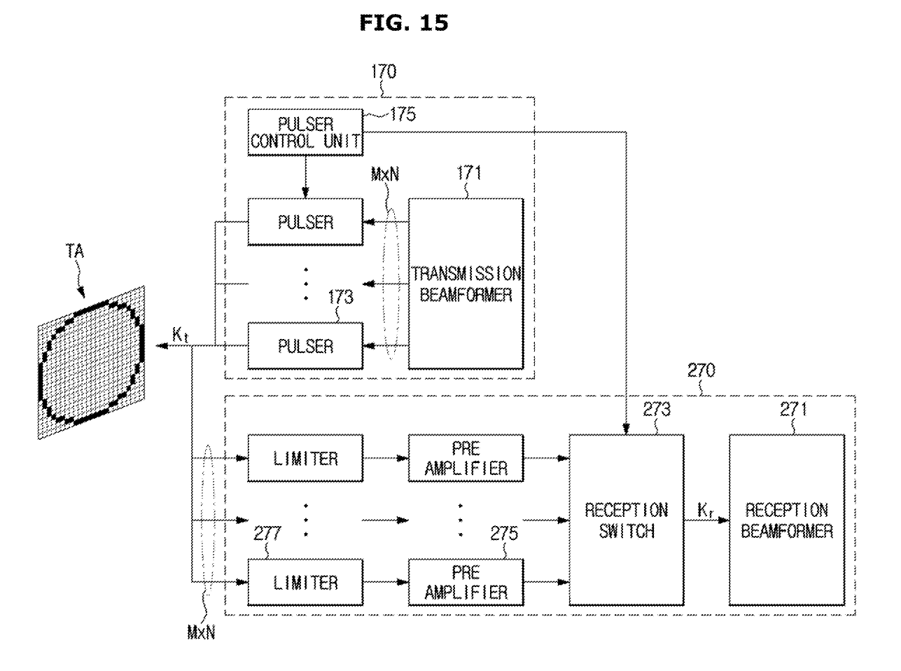

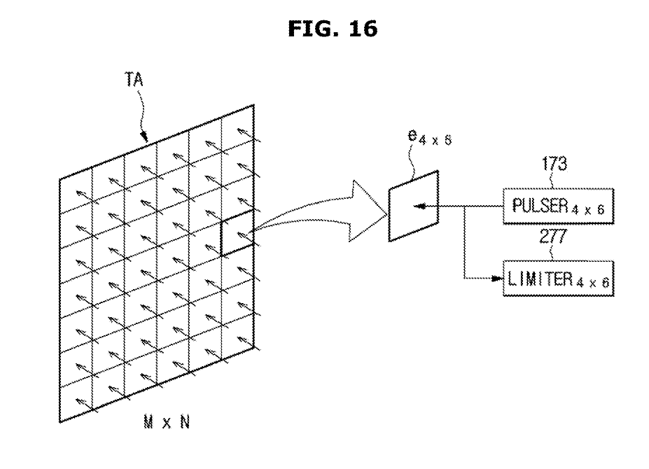

FIG. 14 is a block diagram illustrating a configuration of an ultrasound diagnostic apparatus according to yet another exemplary embodiment. FIGS. 15 and 16 are views illustrating detailed configurations of a transmitter and receiver of an ultrasound probe according to yet another exemplary embodiment. A main body M of the ultrasound diagnostic apparatus 1 according to the present exemplary embodiment has the same configuration as the main bodies M of the aforementioned exemplary embodiments, and so a detailed description thereof will be omitted.

The ultrasound probe P according to the present exemplary embodiment includes a transducer array TA, a transmitter 170, and a receiver 270.

The ultrasound probe P according to the present exemplary embodiment includes neither a T/R switch adjusting a transmitted or received state nor a separate transmission switch selecting elements e when ultrasound waves are transmitted. The number of pulsers 173 according to the present exemplary embodiment is M×N (where M and N are the natural numbers) so as to be respectively connected to the M×N elements e. In other words, the pulsers 173 are connected to the respective elements e. The pulsers 173 function as a switch capable of selecting the elements e to which transmission pulses are to be applied and also function to generate the transmission pulses. When the pulsers 173 are turned on, the transmission pulses are applied to the elements e connected to the corresponding pulsers 173. When the pulsers 173 are turned off, the transmission pulses are not applied to the elements e connected to the corresponding pulsers 173. The pulsers 173 select the elements e such that the elements e to which transmission pulses are applied can form a predetermined shape of aperture AP. The pulsers 173 connected to the elements e determining the shape of the aperture AP to be realized are turned on, and the pulsers 173 connected to the other elements are turned off. Thereby, a specific shape of aperture AP can be realized.

Since the ultrasound probe P according to the present exemplary embodiment does not include the T/R switch, even in a transmitted state in which high-voltage transmission pulses are applied by the pulsers 173, connection between a reception circuit and a transmission circuit including the pulsers 173 is not released. When the connection between the reception circuit and the transmission circuit is not released in the transmitted state, a high voltage from the transmission circuit in the transmitted state is applied to the reception circuit, and a failure may occur at the reception circuit. For this reason, as illustrated in FIG. 15, the ultrasound probe P according to the present exemplary embodiment includes limiters 277 that are connected to the transmission circuit including the pulsers 173 so as to be able to interrupt the high voltage in order to prevent the failure from occurring at the reception circuit due to the high voltage applied from the transmission circuit in the transmitted state. Each limiter 277 is an amplitude limiter that restricts an upper limit of a voltage using a diode, and may be implemented in such a way that a peak form in which a peak clipper and a base clipper are combined.

As illustrated in FIGS. 15 and 16, like the pulsers 173, the limiters 277 are also connected to all of the M×N elements e, and interrupt the high voltage occurring in the transmitted state, thereby protecting the receiver 270. FIG. 16 illustrates connection of the pulser and the limiter to each element e. Since the receiver 270 can be protected from the applied high voltage by the limiters 277, the reception switch 273 of the receiver 270 can be realized using a low-voltage multiplexer (LV MUX). Thus, the ultrasound probe P according to the present exemplary embodiment can omit the T/R switch, and a low-voltage switch can be used as the reception switch 273 in the receiver 270 in spite of omitting the T/R switch. This is favorable in the aspect of a manufacturing cost and realization of the ultrasound probe P.

Hereinafter, the transmitter 170 including the pulsers 173 performing a function of the aforementioned switch together and the receiver 270 including the limiters 277 for interrupting the high voltage will be described in greater detail.

Referring to FIG. 15, the transmission beamformer 171 outputs the M×N transmission signals subjected to transmission beamforming. The M×N time-delayed transmission signals output from the transmission beamformer 171 are input to the pulsers 173. As described above, the pulsers 173 according to the present exemplary embodiment are connected to all the elements e constituting the transducer array TA. The pulser control unit 175 controls on/off of each pulser 173 such that transmission pulses can be applied to Kt elements e forming a predetermined shape of aperture AP among the elements e. For example, the pulser control unit 175 may control the pulsers 173 such that the aperture AP formed of the elements selected by the pulsers 173 can have a ring shape. The shape of the aperture AP is selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to the pulser control unit 175 of the ultrasound probe P. The pulser control unit 175 controls on/off of each pulser 173 on the basis of the information transmitted from the control unit 500 of the main body M, thereby determining the shape of the aperture AP. Without the control of the control unit 500 of the main body M, the pulser control unit 175 may directly control the pulsers 173 in response to input of a command to select the shape of the aperture AP. Although there is no limitation on a shape in which the aperture AP can be realized, like the aforementioned exemplary embodiments, the present exemplary embodiment may control the pulsers 173 such that the aperture AP has a circular or oval shape, or a ring shape so as to be able to maintain isotropy of a resolution in a space.

When the high-voltage transmission pulses are output from the pulsers 173 turned on by the pulser control unit 175, the limiters 277 installed on the respective elements e along with the pulsers 173 interrupt high-voltage signals to prevent the receiver 270 from being destroyed or damaged by the high voltage.

The ultrasound echo signals reflected back from the focal point are input to the transducer array TA, and the transducer array TA converts the input ultrasound echo signals into electric signals.

The electric signals converted by the transducer array TA are amplified by pre amplifiers 275 connected to the respective elements e along with the limiters 277. The M×N electric signals amplified by the pre amplifiers 275 are input to the reception switch 273. Although only the pre amplifiers 275 are illustrated in the figure, the receiver 270 may further include elements capable of adjusting a gain or compensating for attenuation according to a depth before signal processing or time delay processing with respect to the electric signals.

The reception switch 273 selects signals, the number of which is Kr smaller than M×N, from the M×N signals input via the aforementioned pre amplifiers 275. The reception switch 273 selects the signals, the number of which is Kr smaller than M×N that is the number of elements e. Thereby, the ultrasound diagnostic apparatus 1 according to the exemplary embodiment can obtain a three-dimensional volume image with only as many channels as the ultrasound diagnostic apparatus 1 using the one-dimensional array transducer has, despite using the two-dimensional array transducer. The aforementioned pulser control unit 175 controls an operation of the reception switch 273 such that the elements e generating the Kr signals selected by the reception switch 273 along with the control of the pulsers 173 form a predetermined shape of aperture AP. In the present exemplary embodiment, the pulser control unit 175 controls the operation of the reception switch 273. However, a separate reception control unit for controlling the operation of the reception switch 273 may be provided for the receiver 270.

The reception switch 273 selects the electric signals generated from the Kr elements e of the M×N elements e under the control of the pulser control unit 175 so as to be input to the reception beamformer 271. The pulser control unit 175 controls switching of the reception switch 273 such that the Kr elements e selected by the reception switch 273 can form a predetermined shape of aperture AP. For example, the pulser control unit 175 may control the reception switch 273 such that the aperture AP formed of the elements e selected by the reception switch 273 can have a ring shape. The Kr elements e selected by the reception switch 273 may be equal to the Kt elements e selected by the pulsers 173.

The shape of the aperture AP is selected through the input unit 540 by a user, or the shape of the aperture AP may be automatically selected when a specific mode is selected. If a specific shape of aperture AP is selected, the control unit 500 of the main body M may transmit information associated with the shape of the selected aperture AP to the pulser control unit 175 of the ultrasound probe P. The pulser control unit 175 controls an operation of the reception switch 273 on the basis of the information transmitted from the control unit 500 of the main body M, thereby determining the shape of the aperture AP. Without the control of the control unit 500 of the main body M, the pulser control unit 175 may directly control the reception switch 273 in response to input of a command to select the shape of the aperture AP. Although there is no limitation on a shape in which the aperture AP can be realized, an exemplary embodiment may control the reception switch 273 such that the aperture AP has a circular or oval shape, or a ring shape, so as to be able to maintain isotropy of a resolution in a space. The description of the shape of the aperture AP will be omitted because is the description may be identical to the description made with reference to FIG. 8.

The reception beamformer 271 performs beamforming on the electric signals input from the reception switch 273. The reception beamformer 271 strengthens signal intensity by superpositioning the electric signals input from the reception switch 273. When the ultrasound waves of the same phase arrive at the focal point by performing the transmission beamforming, the ultrasound echo signals are generated from the focal point, and then return to the transducer array TA. Similar to when the ultrasound waves are transmitted to the focal point, when the ultrasound echo signals are received from the focal point, distances from the transducer elements e to the focal point are different from each other. As a result, the ultrasound echo signals are different in arrival time from each other. In detail, the ultrasound echo signal arrives at the element nearest the focal point first, and arrives at the element farthest from the focal point last. Since magnitudes of the ultrasound echo signals are very small, it is difficult to obtain necessary information only from the single signal received by each element e. Similar to the transmission beamforming, the reception beamforming is configured to give a proper time delay to the signals that arrive at the respective elements e with a time lag, and to sum up the signals at the same time, thereby improving a signal-to-noise ratio.

The signals beamformed by the reception beamformer 271 are converted into digital signals by an AD converter, and are transmitted to the image processing unit 530 of the main body M. When the AD converter is provided for the main body M, the analog signals beamformed by the reception beamformer 271 may be transmitted to the main body M, and may be converted into the digital signals at the main body M. Alternatively, the reception beamformer 271 may be a digital beamformer. In this case, the digital beamformer may include a storage capable of sampling and storing an analog signal, a sampling period control unit capable of controlling a sampling period, an amplifier capable of adjusting a size of a sample, an anti-aliasing low pass filter for preventing aliasing before the sampling, a band-pass filter capable of selecting a desired frequency band, an interpolation filter capable of increasing a sampling rate in the event of beamforming, and a high-pass filter capable of removing a direct current (DC) component or a low-frequency band signal.

In the disclosed exemplary embodiment, it has been described that the signals go through the reception beamformer 271 once. However, the signals may go through the reception beamformer 271 two or more times so as to further reduce the number of channels. The signals are output from the reception beamformer 271 after the input signals are summed up, and thus the number of signals output from the reception beamformer 271 is smaller than the number of input signals. Therefore, the number of channels can be reduced by causing the signals to go through the reception beamformer 271 several times.

According to the disclosed beamforming apparatus, the ultrasound probe having the beamforming apparatus, and the ultrasound diagnostic apparatus having the beamforming apparatus, the ultrasound waves are transmitted or received to or from some elements that constitute the transducer and form a specific aperture. Thereby, problems that the number of channels caused by using a two-dimensional array transducer is suddenly increased and the resulting complexity of the apparatus is increased can be solved.

Further, the shape of the aperture is controlled so as to be able to maintain the same beam pattern on any region in a space, and thereby the isotropy of the resolution in the space can be maintained.

Although a few exemplary embodiments have been shown and described, it would be appreciated by those skilled in the art that changes may be made in these exemplary embodiments without departing from the principles and spirit of the exemplary embodiments, the scope of which is defined in the claims and their equivalents.

What is claimed is:

1. A beamforming apparatus configured to beamform ultrasound waves transmitted through an ultrasound transducer having a two-dimensional transducer array, the beamforming apparatus comprising:
a transmitter configured to output transmission pulses for driving elements constituting the transducer array; and
a transmission switch configured to select at least two elements among the elements to form an aperture such that the transmission pulses drive the elements forming the aperture.

2. The beamforming apparatus according to claim 1, wherein the transducer array comprises M×N elements, and a number of the selected elements is smaller than (M×N)/2, where M and N are natural numbers.

3. The beamforming apparatus according to claim 1, wherein the transmission switch is configured to select the elements such that the aperture has a ring shape.

4. The beamforming apparatus according to claim 3, wherein the transmission switch is configured to select the elements such that a position at which the ring-shaped aperture is formed on the transducer array varies over time.

5. The beamforming apparatus according to claim 3, wherein the transmission switch is configured to select the elements such that the ring-shaped aperture rotates over time.

6. The beamforming apparatus according to claim 1, wherein the transmission switch is configured to select the elements such that the aperture has a circular shape or a polygonal shape.

7. The beamforming apparatus according to claim 6, wherein the transmission switch is configured to select the elements such that a position at which the circular or polygonal aperture is formed on the transducer array varies over time.

8. The beamforming apparatus according to claim 6, wherein the transmission switch is configured to select the elements such that the circular or polygonal aperture rotates over time.

9. The beamforming apparatus according to claim 1, wherein the transmission switch is configured to select the elements such that the aperture has a linear shape.

10. The beamforming apparatus according to claim 9, wherein the transmission switch is configured to select the elements such that a position at which the linear aperture is formed on the transducer array varies over time.

11. The beamforming apparatus according to claim 9, wherein the transmission switch is configured to select the elements such that the linear aperture rotates over time.

12. The beamforming apparatus according to claim 1, wherein the transmitter comprises:
a transmission beamformer configured to generate transmission signals and add a delay time to the transmission signals and thereby form a transmission signal pattern; and
a pulser configured to generate the transmission pulses configured to drive the elements constituting the transducer array according to the transmission signal pattern.

13. The beamforming apparatus according to claim 12, wherein the transmission beamformer and the pulser are provided plurally, and a number of the transmission beamformers and the pulsers is smaller than a number of the elements constituting the transducer array.

14. The beamforming apparatus according to claim 1, further comprising a transmission and reception switch configured to determine a transmission or reception state of the transducer array.

15. The beamforming apparatus according to claim 1, wherein the transmission switch comprises a high-voltage multiplexer.

16. A beamforming apparatus comprising:
a transmitter configured to output transmission pulses to at least two elements among elements constituting a transducer having a two-dimensional transduce array, the at least two elements forming an aperture;
a receiver configured to focus ultrasound echo signals received by the at least two elements and thereby form a reception beam; and
limiters connected to the respective elements constituting the transducer array and configured to protect the receiver from a high voltage occurring when the transmitter is driven.

17. The beamforming apparatus according to claim 16, wherein the transducer array comprises M×N elements, and a number of the at least two elements forming the aperture is smaller than (M×N)/2, where M and N are natural numbers.

18. The beamforming apparatus according to claim 16, wherein the transmitter comprises:
a transmission beamformer configured to generate transmission signals and add a delay time to the transmission signals to thereby form a transmission signal pattern; and
pulsers that are connected to the respective elements constituting the transducer array and are configured to apply the transmission pulses to the respective elements according to the transmission signal pattern.

19. The beamforming apparatus according to claim 16, wherein the receiver comprises:
pre amplifiers configured to amplify the ultrasound echo signals;
a reception switch configured to select the at least two elements forming the aperture; and
a reception beamformer configured to focus the ultrasound echo signals and thereby form the reception beam, wherein the reception switch comprises a low-voltage multiplexer.

20. The beamforming apparatus according to claim 18, further comprising a pulser controller configured to select and drive only some of the pulsers connected to the elements forming the aperture and control the reception switch such that the reception switch selects the elements forming the aperture.
